(19) United States
(12) Patent Application Publication
(10) Pub. No.: US 2025/0205294 A1
(43) Pub. Date: Jun. 26, 2025

(54) COMPOSITION FOR AMELIORATING OBESITY OR LIPID METABOLISM COMPRISING ASIMINA TRILOBA EXTRACT

(71) Applicant: KOREA FOOD RESEARCH INSTITUTE, Wanju-gun, Jeollabuk-do (KR)

(72) Inventors: Sang Yoon CHOI, Seoul (KR); Chang Jun LEE, Gimhae-si (KR); Jin Young HUR, Yongin-si (KR); Guijae YOO, Jeonju-si (KR); Hyun Hee LEE, Daejeon (KR); Yoon Sook KIM, Wanju-gun (KR); In Wook CHOI, Seoul (KR)

(73) Assignee: KOREA FOOD RESEARCH INSTITUTE, Wanju-gun, Jeollabuk-do (KR)

(21) Appl. No.: 18/852,038

(22) PCT Filed: Dec. 20, 2022
(86) PCT No.: PCT/KR2022/020842

§ 371 Date: Sep. 27, 2024

(30) Foreign Application Priority Data
Mar. 31, 2022 (KR) 10-2022-0040310
Nov. 22, 2022 (KR) 10-2022-0157177

(51) Int. Cl. A61K 36/185; A61P 3/04

(52) U.S. Cl. CPC A61K 36/185; A61P 3/04; A61K 2236/333

(57) ABSTRACT

The present invention relates to a composition for ameliorating, treating or preventing obesity or lipid metabolism, or metabolic diseases, comprising an Asimina triloba extract. The Asimina triloba extract according to the present invention has the effect of ameliorating, preventing or treating obesity, lipid metabolism, or lipid-related metabolic diseases, and thus, a pharmaceutical composition, a food composition, a health functional food, a quasi-drug composition, and a feed composition, while comprise the Asimina triloba extract as an active ingredient, can be effectively used for the prevention, treatment or amelioration of obesity.

### TECHNICAL FIELD

The present disclosure relates to a composition for ameliorating, treating, and preventing obesity or lipid metabolism, or metabolic diseases, comprising an Asimina triloba extract.

### BACKGROUND ART

Obesity generally refers to a state in which adipose tissue is excessively accumulated in the body, and is a phenomenon in which excess energy is accumulated as body fat because the energy intake from food is not balanced with the energy consumed through physical activity, etc. Obesity is known to be caused by a wide variety of causes such as genetic factors, environmental influences due to irregular eating habits, lack of exercise, or westernized diets, psychological influences due to depression, boredom, and excessive stress, and pathological factors due to changes in thyroid or adrenal cortex hormones. Recently, there have been many changes in eating habits due to economic growth and lifestyle changes, and modern people who are busy are increasingly overweight and obese due to high-calorie diets such as fast food and little exercise.

In addition, obesity is caused by hypertrophy of adipocyte cells, and thus the inhibition of adipocyte cell differentiation may be helpful in treating obesity. Previously, anti-obesity studies related to the inhibition of adipocyte cell differentiation and lipid accumulation have been reported.

Obesity has been gradually seriously recognized because of several complications that can be caused by obesity rather than its own danger. Abnormally high levels of body fat due to energy imbalance over a long period of time, can lead to various metabolic diseases and adult diseases such as diabetes, hyperlipidemia, heart disease, stroke, arteriosclerosis, and fatty liver. In addition, obesity has become a serious social problem not only in the West but also in Korea because it can cause mental diseases such as social isolation or alienation, lack of confidence, and depression, as well as physical diseases, and the need for the prevention and treatment of obesity has been recognized as very important.

Obesity can be treated through diet, regular exercise, and lifestyle improvements such as behavioral therapy, as well as medications such as appetite suppressants and fat absorption inhibitors. Since obesity is a chronic disease, long-term use is required when drug treatment is attempted. Currently, products approved for long-term use of 3 months or more in Korea include sibutramine, which is an appetite suppressant, and orlistat, which is a lipase inhibitor. However, most of these anti-obesity drugs are psychotropic drugs that act on the central nervous system to control appetite, so they have side effects such as headaches and vomiting, and have the potential for abuse. Therefore, research is urgently needed to develop a material with high stability and excellent anti-obesity effects that can address the side effects of the above commercially available anti-obesity drugs.

Against this background, the inventors of the present disclosure have made extensive research efforts to develop a naturally derived material useful for the prevention or treatment of obesity, and as a result, have confirmed that an Asimina triloba extract has an effect of inhibiting lipid production and may be applied as an anti-obesity material, thereby completing the present disclosure.

### DISCLOSURE

### Technical Problem

An object of the present disclosure is to solve the foregoing problems and other problems related thereto.

One exemplary object of the present disclosure is to provide a food composition for preventing or ameliorating obesity or lipid metabolism, or metabolic diseases, comprising an Asimina triloba extract as an active ingredient.

Another exemplary object of the present disclosure is to provide a pharmaceutical composition for preventing or treating obesity or metabolic diseases, comprising an Asimina triloba extract as an active ingredient.

Another exemplary object of the present disclosure is to provide a health functional food for preventing or ameliorating obesity, lipid metabolism, or metabolic diseases, comprising an Asimina triloba extract as an active ingredient.

Another exemplary object of the present disclosure is to provide a quasi-drug composition for preventing, ameliorating, or treating obesity or metabolic diseases, comprising an Asimina triloba extract as an active ingredient.

Another exemplary object of the present disclosure is to provide a feed composition for preventing or ameliorating obesity or lipid metabolism, or metabolic diseases, comprising an Asimina triloba extract as an active ingredient.

Another exemplary object of the present disclosure is to provide a method for preventing or treating obesity or lipid metabolism, or metabolic diseases, comprising administering to a subject a composition comprising an Asimina triloba extract as an active ingredient.

Another exemplary object of the present disclosure is to provide use of an Asimina triloba extract or a composition comprising the Asimina triloba extract for the prevention or treatment of obesity or lipid metabolism, or metabolic diseases.

Another exemplary object of the present disclosure is to provide use of an Asimina triloba extract or a composition comprising the Asimina triloba extract for the preparation of a medicament for the prevention or treatment of obesity or lipid metabolism, or metabolic diseases.

Another exemplary object of the present disclosure is to provide a method for inhibiting differentiation of preadipocyte cells and inhibiting expression of transcription factors involved in adipogenesis and preadipocyte cell differentiation by administering an Asimina triloba extract.

The technical challenges to be achieved according to the technical idea of the present disclosure disclosed in the present specification are not limited to the challenges for solving the above-mentioned problems, and additional challenges not mentioned will be clearly understood by those skilled in the art from the following description.

### Technical Solution

This will be specifically described as follows. Meanwhile, each of f the descriptions and embodiments disclosed in the present application may also be applied to each other descriptions and embodiments. That is, all combinations of various elements disclosed in the present application fall within the scope of the present application. In addition, the scope of the present application is not to be considered limited by the specific description set forth below.

As one aspect for achieving the above object, the present disclosure provides a food composition for preventing or ameliorating obesity, lipid metabolism, or metabolic diseases, comprising an Asimina triloba extract as an active ingredient.

As used herein, “Asimina triloba” is a deciduous shrub of the Asimina triloba family, which is mainly native to the eastern United States and Canada, and is a plant that is easy to grow due to its strong resistance to diseases and pests and cold. Although Asimina triloba is known to have anticancer components, but its use related to obesity has not yet been known.

As used herein, Asimina triloba may be used as it is without damage to its original form, or may be used after performing a preprocessing process in consideration of a process speed and process (manufacturing) efficiency intended by those skilled in the art. As the preprocessing process, for example, steps such as conventional screening, washing with water, cutting, powdering, drying, etc. may be performed.

As used herein, “obesity” refers to a state in which adipocyte cells proliferate and differentiate in the body, resulting in excessive accumulation of adipose, and may cause related complications, including metabolic diseases accompanied by hypertension, diabetes, and dyslipidemia, etc. When energy intake increases relative to energy consumption, the number and volume of adipocyte cells increase, resulting in an increase in the mass of adipose tissue.

As used herein, “ameliorating lipid metabolism” may include suppressing intracellular lipid accumulation, improving blood lipids, reducing weight, reducing body fat, or ameliorating blood cholesterol, etc.

As used herein, “metabolic diseases” may be lipid-related metabolic diseases, and may be one or more selected from the group consisting of, but not limited to, for example, diabetes, diabetic complications, stroke, hyperlipidemia, fatty liver, arteriosclerosis, hypertension, cardiovascular diseases, or metabolic syndrome in which the above diseases occur simultaneously.

The diabetic complications may be, but are not limited to, diabetic neuropathy, diabetic nephropathy, diabetic myocardial infarction, diabetic retinopathy, diabetic cataract, or diabetic foot ulcer.

As used herein, an “extract” includes an extract itself and an extract of any formulation that may be formed using the extract such as the extract obtained by extracting the Asimina triloba, a diluted or concentrated solution of the extract, a dried product obtained by drying the extract, a crude purified product or purified product of the extract, or a mixture thereof.

As used herein, the extraction is not particularly limited, and may be carried out according to a method commonly used in the art. Non-limiting examples of the extraction method include hot water extraction, cold sediment extraction, solvent extraction, steam distillation, elution, expression, ultrasonic extraction, filtration, reflux extraction, etc., and these may be carried out alone or in combination of two or more methods.

The extract of the present disclosure is an extract obtained by extracting the Asimina triloba using an appropriate solvent, and may include, for example, a crude extract, a polar solvent-soluble extract, or a non-polar solvent-soluble extract. The type of the extraction solvent used to prepare the extract is not particularly limited, and any solvent known in the art may be used. Non-limiting examples of the extraction solvent may include water; alcohols having 1 to 4 carbon atom such as methanol, ethanol, propanol, isopropanol, butanol, propyl alcohol, and butyl alcohol; polyhydric alcohols such as glycerin, butylene glycol, and propylene glycol; hydrocarbon-based solvents such as methyl acetate, ethyl acetate, acetone, benzene, hexane, diethyl ether, and dichloromethane; or a mixture thereof, and for example, a solvent selected from the group consisting of water, an alcohol having 1 to 4 carbon atoms, or a mixed solvent thereof. An example of the extraction solvent may be a solvent selected from the group consisting of water, an alcohol having 1 to 4 carbon atoms, or a mixed solvent thereof.

As used herein, the Asimina triloba extract may be one or more extracts selected from the group consisting of the seeds, fruits, roots, stems, and leaves of Asimina triloba, and may be, for example, an Asimina triloba fruit extract.

As used herein, the Asimina triloba extract may inhibit the differentiation of preadipocyte cells and inhibit expression of transcription factors involved in adipogenesis and preadipocyte cell differentiation. Examples of such transcription factors include, but are not limited to, FAS, PPARγ, C/EBPα, and adiponectin.

Specifically, it was confirmed from the embodiment of the present disclosure that when the Asimina triloba extract was treated with differentiation-induced preadipocyte cell 3T3-L1, adipogenesis was inhibited and the expression of FAS, PPARγ, C/EBPα, and adiponectin, which are involved in adipogenesis or preadipocyte cell differentiation induction, was significantly reduced.

The food composition according to the present disclosure may be prepared in various forms of formulations, and has the advantage that there are no side effects that may occur during long-term use of medication because food is used as a raw material, unlike general drugs. The food composition according to the present disclosure may be prepared in any form, and may be specifically formulated into one or more formulations selected from, but not limited to, the group consisting of health functional food preparations such as tablets, capsules, pills, granules, liquids, powders, flakes, pastes, syrups, gels, jellies, and bars, beverages, chewing gums, and candies.

In addition, the food composition according to the present disclosure may comprise food additives in addition to the active ingredients. The food additives may be generally understood as substances that are mixed or infiltrated by being added to food in preparing, processing, or preserving food, and their safety needs to be ensured because they are consumed daily and over a long period of time with food. The food additives are classified into sweeteners, flavoring agents, preservatives, emulsifiers, acidulants, thickeners, etc. in terms of function, and are not particularly limited as long as they are consistent with the purpose to be achieved by the food composition according to the present disclosure. In addition, the food composition according to the present disclosure may comprise, in addition to the food additives, physiologically active substances or minerals known in the art for functional and nutritional supplementation and whose stability as food additives is assured. The physiologically active substances or minerals are not particularly limited as long as they are consistent with the purpose to be achieved by the food composition according to the present disclosure.

The food composition according to the present disclosure may comprise the foregoing food additives in an effective amount capable of achieve the purpose for which they are added, depending on the type of the product, and with respect to other food additives that may be included in the food composition according to the present disclosure, reference may be made to the food codes or food additive codes of each country.

Another aspect of the present disclosure for achieving the above purpose provides a pharmaceutical composition for preventing or treating obesity or lipid-related metabolic diseases, comprising an Asimina triloba extract as an active ingredient.

As used herein, “Asimina triloba”, “extract”, “obesity”, or “metabolic diseases” are as described above.

As used herein, “prevention” refers to any action by which obesity or lipid-related metabolic diseases are suppressed or delayed by administrating the pharmaceutical composition according to the present disclosure. As used herein, “treatment” refers to any action by which obesity or lipid-related metabolic diseases that have developed are improved, alleviated, or beneficially changed by administrating the pharmaceutical composition according to the present disclosure. As used herein, “amelioration” refers to any action by which obesity or lipid-related metabolic diseases are ameliorated by administration the pharmaceutical composition according to the present disclosure.

As used herein, the “pharmaceutical composition” refers to a composition prepared for the purpose of preventing or treating a disease, each of which may be formulated and used in various forms according to conventional methods. For example, the pharmaceutical composition may be formulated into oral formulations such as powders, granules, tablets, capsules, suspensions, emulsions, and syrups, depending on the administration route, and may be formulated and used in the form of external preparations and sterile injectable solutions. Specifically, the administration route may be any appropriate route, including a topical route, an oral route, an intravenous route, an intramuscular route, and direct absorption through mucosal tissue, and may also be used in combination of two or more routes. An example of a combination of two or more routes is a combination of two or more drug formulations according to the administration route, for example, where one drug is primarily administered via the intravenous route and the other drug is secondarily administered via the topical route.

The pharmaceutical composition according to the present disclosure may further comprise pharmaceutically acceptable carriers, excipients, or diluents according to a conventional method. Pharmaceutically acceptable carriers are well known in the art, depending on the administration route or formulation, and specifically, reference can be made to the pharmacopoeias of each country including the ‘Korean Pharmacopoeia.’ Carriers, excipients, and diluents that may be included in the composition according to the present disclosure include, but are not limited to, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil. In addition, carriers, excipients, and diluents that may be included in the composition according to the present disclosure may be an unnatural carrier, but the present disclosure is not limited thereto.

The pharmaceutical composition according to the present disclosure may be formulated and used in the form of oral formulations such as powders, granules, tablets, capsules, suspensions, emulsions, syrups, and aerosols, external preparations, suppositories, or sterile injection solutions according to a conventional method. Specifically, the pharmaceutical composition may be formulated, diluents or excipients such as fillers, extender, binders, wetting agents, disintegrants, and surfactants that are commonly used, may be used. Solid preparations for oral administration include tablets, pills, powders, granules, capsules, etc., and such solid preparations may be prepared by mixing the compound with at least one excipient, for example, starch, calcium carbonate, sucrose, lactose, gelatin, etc. Further, in addition to simple excipients, lubricants such as magnesium stearate and talc may also be used. Liquid preparations for oral administration include suspensions, oral liquids, emulsions, syrups, etc., and may include various excipients, for example, wetting agents, sweeteners, fragrances, preservatives, etc., in addition to commonly used simple diluents such as water and liquid paraffin. Preparations for parenteral administration include sterile aqueous solutions, non-aqueous solvents, suspensions, emulsions, lyophilized preparations, and suppositories. As non-aqueous solvents and suspensions, propylene glycol, polyethylene glycol, vegetable oils such as olive oil, and injectable esters such as ethyl oleate may be used. As a base for the suppositories, Witepsol, macrogol, Tween 61, cacao butter, laurin butter, glycerol gelatin, etc. may be used. Specific formulations of pharmaceutical compositions are known in the art and can be found, for example, in Remington's Pharmaceutical Sciences (19th ed. 1995), which is incorporated herein by reference.

The pharmaceutical composition according to the present disclosure is administered in a pharmaceutically effective amount. The pharmaceutically effective amount refers to an amount that is sufficient to treat a disease at a reasonable benefit/risk ratio applicable to medical treatment and is unlikely to cause side effects. An effective dose level may be determined based on factors including patient's health condition, type and severity of the disease, activity of the drug, sensitivity of the drug, administration methods, administration time, administration route, and excretion rate, treatment periods, drugs used in combination or simultaneously, and other factors well known in the medical field. The dosage and frequency of administration do not limit the scope of the present disclosure in any way.

The pharmaceutical composition according to the present disclosure may be administered to mammals such as mouse, dog, cat, cow, horse, pig, and human, and preferably human, through various routes. Any mode of administration may be expected, and includes, but is not limited to, for example, oral, intravenous, intramuscular, or subcutaneous injection.

As used herein, the phrase “comprising as an active ingredient” means that the pharmaceutical composition comprises an effective amount of the active ingredient in an amount sufficient to produce a preventive or therapeutic effect on obesity or metabolic diseases.

Another aspect of the present disclosure for achieving the above purpose provides a health functional food for preventing or ameliorating obesity, lipid metabolism, or metabolic diseases, comprising an Asimina triloba extract.

As used herein, the Asimina triloba, the extract, obesity, or metabolic disease are as described above.

As used herein, the term “health functional food” refers to a food that is prepared and processed using raw materials or ingredients that have functionality useful to the human body. The “functionality” means obtaining a useful effect on the structure and function of the human body for health applications such as controlling nutrients or physiological actions. The health functional food according to the present disclosure may be prepared by a method commonly used in the art, and may be prepared by adding raw materials and ingredients commonly added in the art during the preparation.

Furthermore, the formulation of the health functional food may also be prepared without limitation as long as it is a formulation recognized as a health functional food. Non-limiting examples of the formulation may include, but are not limited to, one or more formulations selected from the group consisting of health functional food preparations such as tablets, capsules, pills, granules, liquids, powders, flakes, pastes, syrups, gels, jellies, and bars, beverages, chewing gums, and candies.

Another aspect of the present disclosure for achieving the above object provides a quasi-drug composition for preventing, ameliorating, or treating obesity or metabolic diseases, comprising an Asimina triloba extract as an active ingredient.

The quasi-drug composition according to the present disclosure may comprise, in addition to the Asimina triloba extract, ingredients commonly used in the quasi-drug composition such as, but not limited to, abrasives, wetting agents, binders, foaming agents, sweeteners, preservatives, medicinal ingredients, flavorings, pigments, solvents, brighteners, solubilizers, or pH adjusters.

In the present disclosure, the type of the quasi-drug is not particularly limited, and may be any quasi-drugs commonly used in the art. Non-limiting examples of the quasi-drug may be one or more formulation selected from the group consisting of toothpastes, mouthwashes, chewing gums, candies, oral sprays, oral ointments, oral varnishes, oral films, and gum massage creams. These may be used alone or in combination of two or more.

Another aspect of the present disclosure for achieving the above object provides a feed composition for ameliorating or preventing obesity, lipid metabolism, or metabolic diseases, comprising an Asimina triloba extract as an active ingredient.

As used herein, the Asimina triloba, the extract, obesity, or metabolic disease are as described above.

The feed composition may comprise a feed additive. The feed additive according to the present disclosure corresponds to an auxiliary feed under the Feed Management Act.

As used herein, the term “feed” may refer to any natural or artificial diet, meal, etc., or ingredients of the meal, which is intended or adapted to be eaten, consumed, and digested, in particular by animals.

The type of the feed is not particularly limited, and feed commonly used in the art may be used. Non-limiting examples of the feed include plant-based feeds, such as grains, root crops, food processing by-products, algae, fibers, pharmaceutical by-products, oils, starches, gourds, or grain by-products; and animal feeds such as proteins, inorganics, oils, minerals, oils, single-cell proteins, animal plankton, or food. These may be used alone or in combination of two or more.

Another aspect of the present disclosure for achieving the above purpose provides a method for ameliorating, preventing, or treating obesity or lipid-related metabolic diseases, comprising administering to a subject in need thereof an effective amount of an Asimina triloba extract for ameliorating, preventing, or treating obesity or lipid-related metabolic diseases.

As used herein, the “effective amount” refers to an amount sufficient for the composition according to the present disclosure described above to achieve the effect of ameliorating, preventing, or treating obesity or lipid-related metabolic diseases.

As used herein, term “subject” includes, but is not particularly limited to, human, monkey, cow, horse, sheep, pig, chicken, turkey, quail, cat, dog, mouse, rat, rabbit, or guinea pig, and preferably mammal, and more preferably human.

As used herein, the term “administration” means providing a predetermined material to a subject by any appropriate method.

The administration route of the composition according to the present disclosure may be administered orally or parenterally through any general route as long as it may reach the target tissue. In addition, the composition according to the present disclosure may also be administered using any device capable of delivering the active ingredient to the target cell, tissue, or organ.

Another aspect of the present disclosure for achieving the above object provides a method for preventing or treating obesity or lipid metabolism, or metabolic diseases, comprising administering to a subject a composition comprising an Asimina triloba extract as an active ingredient.

Another aspect of the present disclosure for achieving the above object provides a use of an Asimina triloba extract or a composition comprising the an Asimina triloba extract for preventing or treating obesity or lipid metabolism, or metabolic diseases.

Another aspect of the present disclosure for achieving the above object provides a use of an Asimina triloba extract or a composition comprising the Asimina triloba extract for the preparation of a medicament for the prevention or treatment of obesity or lipid metabolism, or metabolic diseases.

Another aspect of the present disclosure for achieving the above object provides a method for inhibiting differentiation of preadipocyte cells and inhibiting expression of transcription factors involved in adipogenesis and preadipocyte cell differentiation by administering an Asimina triloba extract.

### Advantageous Effects

The Asimina triloba extract according to the present disclosure has the effect of ameliorating, preventing, or treating obesity or metabolic diseases, and thus, a pharmaceutical composition, a food composition, a health functional food, a quasi-drug composition, and a feed composition, which comprise the Asimina triloba extract as an active ingredient, can be effectively used for the prevention, treatment, or amelioration of obesity.

### DESCRIPTION OF DRAWINGS

FIG. 1 illustrates the results of evaluating the cytotoxicity of an Asimina triloba extract.

FIG. 2 illustrates the effect of the Asimina triloba extract on the inhibition of adipocyte cell differentiation in preadipocyte cells (3T3-L1).

FIG. 3 illustrates the results of confirming the protein expression levels of adipogenesis-related transcription factors (FAS, PPARγ, C/EBPα, and adiponectin) in preadipocyte cells after treatment with the Asimina triloba extract.

FIG. 4 illustrates the results of confirming the expression level of AMPK protein in preadipocyte cells after treatment with the Asimina triloba extract.

FIG. 5 illustrates changes in body weight over 12 weeks in a normal group (N), a high-fat diet group (C), a positive control group (P), an Asimina triloba fruit extract group at a low-concentration (AT-L), and an Asimina triloba fruit extract group at a high-concentration (AT-H).

FIG. 6 illustrates the results of analyzing a body fat composition in a normal group (N), a high-fat diet group (C), a positive control group (P), an Asimina triloba fruit extract group at a low-concentration (AT-L), and an Asimina triloba fruit extract group at a high-concentration (AT-H).

FIG. 7 illustrates the results of confirming the weight of adipose tissue in a normal group (N), a high-fat diet group (C), a positive control group (P), an Asimina triloba fruit extract group at a low-concentration (AT-L), and an Asimina triloba fruit extract group at a high-concentration (AT-H).

FIG. 8 illustrates the results of confirming a ratio of fat to a total body weight in a normal group (N), a high-fat diet group (C), a positive control group (P), an Asimina triloba fruit extract group at a low-concentration (AT-L), and an Asimina triloba fruit extract group at a high-concentration (AT-H).

FIG. 9 illustrates the results of confirming the weight of liver in a normal group (N), a high-fat diet group (C), a positive control group (P), an Asimina triloba fruit extract group at a low-concentration (AT-L), and an Asimina triloba fruit extract group at a high-concentration (AT-H).

FIG. 10 illustrates the results of confirming the weight of epididymal fat in a normal group (N), a high-fat diet group (C), a positive control group (P), an Asimina triloba fruit extract group at a low-concentration (AT-L), and an Asimina triloba fruit extract group at a high-concentration (AT-H).

FIG. 11 illustrates the results of confirming the weight of perirenal adipose tissue in a normal group (N), a high-fat diet group (C), a positive control group (P), an Asimina triloba fruit extract group at a low-concentration (AT-L), and an Asimina triloba fruit extract group at a high-concentration (AT-H).

FIG. 12 illustrates the results of visual observation of the liver in a normal group (N), a high-fat diet group (C), a positive control group (P), an Asimina triloba fruit extract group at a low-concentration (AT-L), and an Asimina triloba fruit extract group at a high-concentration (AT-H).

FIG. 13 illustrates the results of observing lipid droplet in stained liver using Oil Red O stain in a normal group (N), a high-fat diet group (C), a positive control group (P), an Asimina triloba fruit extract group at a low-concentration (AT-L), and an Asimina triloba fruit extract group at a high-concentration (AT-H).

FIG. 14 illustrates the results of H&E staining experiments on liver tissue in a normal group (N), a high-fat diet group (C), a positive control group (P), an Asimina triloba fruit extract group at a low-concentration (AT-L), and an Asimina triloba fruit extract group at a high-concentration (AT-H).

FIG. 15 illustrates the results of measuring blood glucose in a normal group (N), a high-fat diet group (C), a positive control group (P), an Asimina triloba fruit extract group at a low-concentration (AT-L), and an Asimina triloba fruit extract group at a high-concentration (AT-H).

FIG. 16 illustrates the results of confirming the blood glucose area under the curve (AUC) in a normal group (N), a high-fat diet group (C), a positive control group (P), an Asimina triloba fruit extract group at a low-concentration (AT-L), and an Asimina triloba fruit extract group at a high-concentration (AT-H).

FIG. 17 illustrates the results of measuring a total cholesterol concentration in a normal group (N), a high-fat diet group (C), a positive control group (P), an Asimina triloba fruit extract group at a low-concentration (AT-L), and an Asimina triloba fruit extract group at a high-concentration (AT-H).

FIG. 18 illustrates the results of measuring a low-density lipoprotein concentration in a normal group (N), a high-fat diet group (C), a positive control group (P), an Asimina triloba fruit extract group at a low-concentration (AT-L), and an Asimina triloba fruit extract group at a high-concentration (AT-H).

FIG. 19 illustrates the results of measuring a high-density lipoprotein concentration in a normal group (N), a high-fat diet group (C), a positive control group (P), an Asimina triloba fruit extract group at a low-concentration (AT-L), and an Asimina triloba fruit extract group at a high-concentration (AT-H).

FIG. 20 illustrates the results of measuring a concentration of aspartate aminotransferase in a normal group (N), a high-fat diet group (C), a positive control group (P), an Asimina triloba fruit extract group at a low-concentration (AT-L), and an Asimina triloba fruit extract group at a high-concentration (AT-H).

FIG. 21 illustrates the results of measuring a concentration of alanine aminotransferase in a normal group (N), a high-fat diet group (C), a positive control group (P), an Asimina triloba fruit extract group at a low-concentration (AT-L), and an Asimina triloba fruit extract group at a high-concentration (AT-H).

FIG. 22 illustrates the results of measuring a concentration of lactate dehydrogenase in a normal group (N), a high-fat diet group (C), a positive control group (P), an Asimina triloba fruit extract group at a low-concentration (AT-L), and an Asimina triloba fruit extract group at a high-concentration (AT-H).

### BEST MODE

Hereinafter, the present disclosure will be described in more detail with reference to the following Examples. However, these Examples are intended to illustrate the present disclosure by way of example, and the scope of the present disclosure is not limited to these Examples.

### Example 1. Preparation of Asimina triloba Extract

Asimina triloba fruits were purchased and used from Geochang, Gyeongsangnam-do, Korea. The Asimina triloba fruits were dried and extracted twice at 40° C. for 3 hours using 70% ethanol (EtOH, the remaining 30% water). After extraction, the extract was concentrated under reduced pressure using a vacuum concentrator and then freeze-dried. The prepared extract was stored at −80° C. and used for experiment.

### Experimental Example 1. In Vitro Analysis of Anti-Obesity Efficacy of Asimina triloba Extract

### 1-1. Cell Culture and Differentiation Induction

In order to confirm the anti-obesity effect of an Asimina triloba extract, 3T3-L1 preadipocyte cells were cultured and differentiation was induced.

Specifically, 3T3-L1 cells were obtained from the American Type Culture Collection (ATCC, USA) and cultured in Dulbecco's Modified Eagle Medium (DMEM, Gibco, USA) containing 10% bovine serum (BS, Gibco, USA) and 1% penicillin-streptomycin (PS, Gibco, USA) under 5% CO2 and at 37° C.

For differentiation induction, 80% confluent cells were seeded at 1×10⁵ cells/well in 6 wells using trypsin-EDTA. After 24 hours, when the cells were post-confluent, differentiation was induced by treating them with 0.5 mM 3-isobutyl-1-methylxanthine (IBMX, Sigma, USA), 10 μg/mL insulin (Sigma, USA), 10 μM dexamethasone (Sigma, USA), and 2 μM rosiglitazone (Sigma, USA) (IBMX, insulin, dexamethasone, and rosiglitazone mixture: denoted as MDI) in DMEM culture medium supplemented with 10% fetal bovine serum. 2 days after differentiation induction, the medium was replaced with 10% FBS DMEM with 10 μg/mL insulin, and the same medium was replaced on days 4 and 6 of differentiation induction to induce adipocyte cell differentiation for a total of 8 days.

All experiments were then expressed as mean values and standard error of the mean for triplicate experiments, and the test of significance between each group was performed by Graphpad PRISM 7 (Graph pad software, USA) using one-way ANOVA and Turkey method, and a p value of less than 0.05 (p<0.05) was considered statistically significant.

### 1-2. Cytotoxicity Analysis

In order to confirm whether an Asimina triloba extract was toxic to preadipocyte cells, the cell viability was measured using 3-(4, 5-Dimethylthiazol-2-yl)-2, 5-Diphenyltetrazolium Bromide (MTT) assay.

Specifically, the differentiation-uninduced 3T3-L1 cells (Natural, N) were seeded at 2×10⁴ cells/well in a 96-well plate, and treated with MDI and an Asimina triloba extract at 50, 100, and 200 μg/mL, respectively, after 24 hours. After 48 hours, the cells were treated with 5 mg/mL of 3-(4, 5-dimethylthiazol-2-yl)-2, 5-diphenyltetrazolium bromide (MTT, Sigma, USA). After 1 hour, the medium was removed and dimethylsulfoxide (DMSO) was treated to dissolve the generated formazan. The absorbance was measured at 570 nm to evaluate the cell viability. The differentiation-uninduced 3T3-L1 cells (Natural, N) were used as a control group.

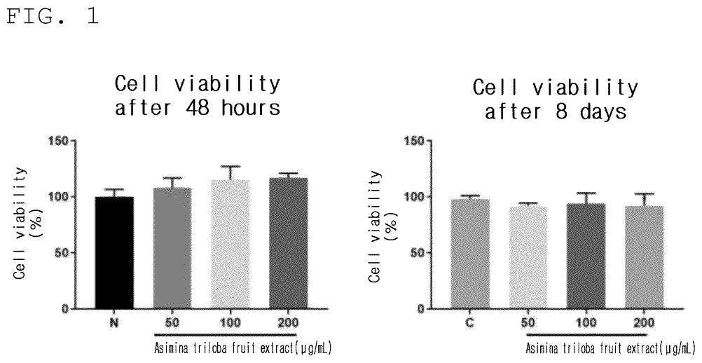

The results showed that the cell viability was similar or higher in the Asimina triloba extract-treated group compared to the control group regardless of the concentration, confirming that the Asimina triloba extract did not exhibit cytotoxicity. In addition, when 3T3-L1 cells were seeded at 2×10⁴ cells/well, the 3T3-L1 cells (Control, C) of which the differentiation was not induced with MDI were treated with the Asimina triloba extract 24 hours later, and the cell viability was evaluated after 8 days, the cell viability was similar in the Asimina triloba extract-treated groups compared to the differentiation-induced 3T3-L1 cells as the control group, regardless of the concentration (FIG. 1), confirming that the Asimina triloba extract was not cytotoxic.

### 1-3. Analysis of Inhibition of Adipogenesis

In order to confirm the inhibitory effect of the Asimina triloba extract on adipogenesis, the differentiation-induced 3T3-L1 cells in Experimental Example 1-1 were treated with the Asimina triloba extract and the fat content was measured.

Specifically, the differentiation-induced 3T3-L1 cells in Experimental Example 1-1 were treated with the Asimina triloba extract at concentrations of 50, 100, and 200 μg/mL, respectively. On day 8, the medium was removed, the cells were washed with phosphate-buffered saline (PBS, Gibco, USA), and fixed with 3.6% formaldehyde for 15 minutes. After washing three times with distilled water, the cells were treated with the filtered Oil Red O solution and stained for 15 minutes. The stained cells were washed three times with distilled water, photographed, and the intracellular stained fat was eluted with 100% isopropanol, transferred to a 96-well plate. Then, the degree of fat accumulation was evaluated by measuring the absorbance at 490 nm.

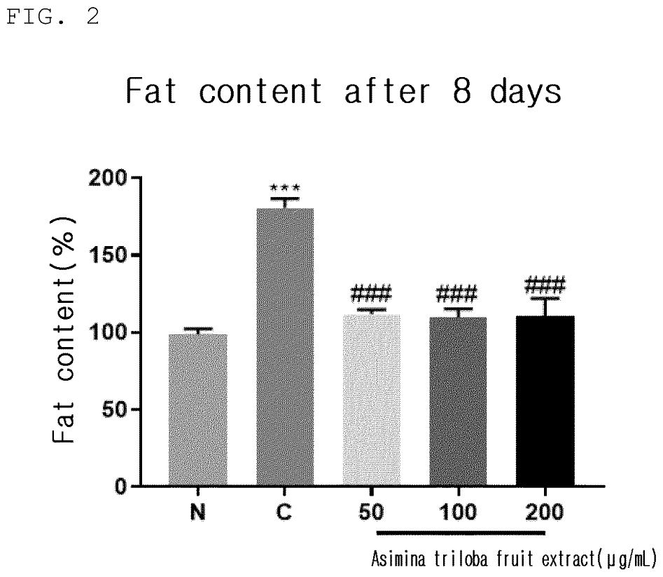

As a result, it was confirmed that the fat content of the lipid droplet in the differentiation-induced 3T3-L1 cells (C) as the control group, was increased by more than 1.8 times compared to the undifferentiated cells (N). When treated with the Asimina triloba extract, the fat content was significantly reduced compared to the differentiation-induced 3T3-L1 cells regardless of the concentration, confirming that the Asimina triloba extract has an inhibitory effect on adipogenesis (FIG. 2).

### 1-4. Analysis of Expression of Adipogenic Proteins

The inhibitory effect of the Asimina triloba extract on adipogenesis was analyzed through the expression levels of representative adipogenic markers, FAS, PPARγ, C/EBPα, and adiponectin. FAS is known to be involved in fatty acid production, PPARY is known to promote fat absorption by regulating fatty acid storage and glucose metabolism, C/EBPα interacts with PPARγ to promote the later stages of differentiation of preadipocyte cells, and adiponectin regulates fatty acid and glucose metabolism and suppresses insulin resistance.

Specifically, in order to confirm the expression of proteins that play a key role in the induction of preadipocyte cells into adipocyte cells, 3T3-L1 cells (Natural, N), the differentiation-induced 3T3-L1 (Control, C) cells in Experimental Example 1-1, and cells treated with 50, 100, and 200 μg/mL of the Asimina triloba extract, respectively, were lysed in PRO-PREP for cell/tissue (iNtRON Biotechnology, Korea) with 1% protease & phosphatase (Thermo Fisher Scientific, USA) to elute the proteins in the cells. The protein extracts were quantified and the amount of protein in all groups was equalized by dilution with distilled water. 4× raemmli sample buffer (Bio-rad, USA) was added to the equal volume of protein extracts, reacted at 100° C. for 5 minutes, and then cooled to prepare Western blot protein samples. The resulting protein samples were electrophoresed using SDS-PAGE (Bio-rad, USA) and then transferred to a polyvinylidene difluoride membrane (Bio-rad, USA). The transferred membrane was blocked in a buffer containing 5% skim milk for 1 hour and then incubated with a primary antibody diluted 1:1000 at 4° C. The next day, HRP-labeled goat anti-rabbit IgG or goat anti-mouse IgG, which is secondary antibody, was diluted 1:5000 and incubated for 1 hour at room temperature. Finally, the membrane was reacted with WesernSure PREMIUM Chemiluminescent Substrate (LI-COR, USA) for 1 minute, and the protein expression level was detected using ChemiDoc (Bio-rad, USA).

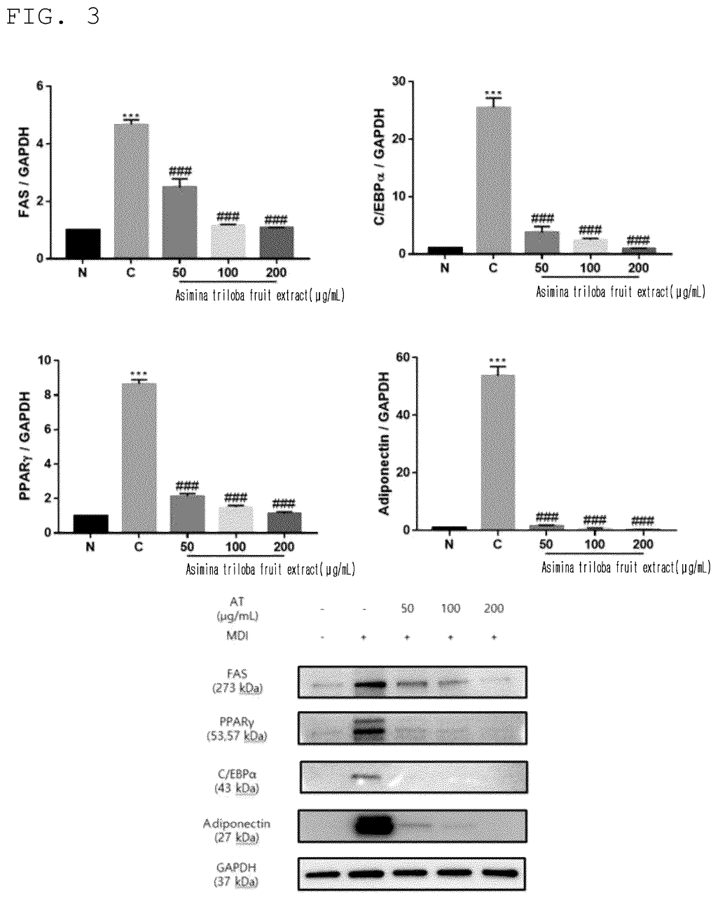

As a result, it was confirmed that the expression levels of FAS, PPARγ, C/EBPα, and adiponectin were significantly reduced compared to the differentiation-induced 3T3-L1 cells when treated with the Asimina triloba extract (FIG. 3).

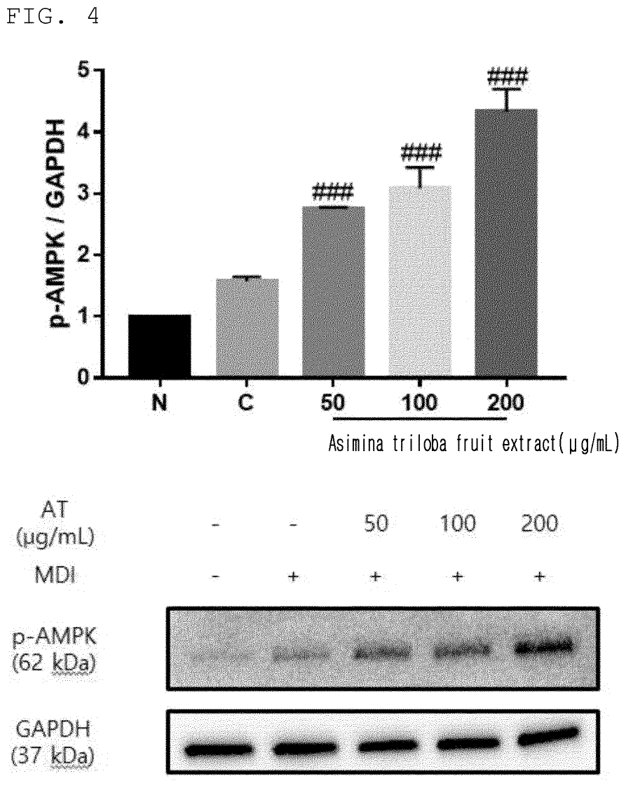

In addition, the expression level of AMP-activated protein kinase (AMPK), which plays an important role in maintaining energy homeostasis in the body by mediating the synthesis and decomposition of fatty acids, was confirmed that the treatment with the Asimina triloba extract activated AMPK compared to the differentiation-induced 3T3-L1 cells, resulting in an inhibitory effect on adipogenesis (FIG. 4).

### Experimental Example 2. Analysis of Anti-Obesity Efficacy of Asimina triloba Extract In Vivo Animal Model

### 2-1. Preparation of the Animal Model

5-week-old male C57BL6 mice (Orient Bio Inc., Seongnam, Republic of Korea) were purchased and maintained at a temperature of 23±2° C., a humidity of 50±10%, and a light/dark cycle of 12 hours, and were fed 10% fat (TD. 06416, ENVIGO, Indianapolis, USA) and water. This animal experiment was conducted with the approval of the Institutional Animal Care and Use Committee (IACUC) of the Korea Food Research Institute.

After a 1-week adaptation period, the mice weighed 19.96±0.70 g on average weight and were divided into 5 groups (n=9 per group, 4 to 5 mice per cage) by the randomized complete block design. The mice were divided into a normal group (N, 10% fat), a high-fat diet group (Control, C, 60% high-fat (TD.06414, ENVIGO, Indianapolis, IN, USA)), a positive control group (Positive, P, 60% high-fat+Garcinia cambogia extract 165 mg/kg body weight), an Asimina triloba fruit group at a low-concentration (60% high-fat+Asimina triloba fruit extract 100 mg/kg body weight, AT-L), an Asimina triloba fruit group at a high-concentration (60% high-fat+Asimina triloba fruit extract 300 mg/kg body weight, AT-H) and orally administered the extract dissolved in saline at a certain time every day using a zonde and were raised for 12 weeks. The body weight and food intake were measured once a week. Before autopsy, blood was collected after a 12-hours fast and tissues were extracted

### 2-2. Body Composition and Tissue Analysis

The body weight, body composition, and tissue of mice in each experimental group were observed to confirm whether the Asimina triloba extract has an inhibitory effect on adipogenesis.

Specifically, mouse body composition was analyzed using dual-energy X-ray absorptiometry (InAlyzer, Medikors Inc., Seongnam, Korea). Anesthetized mice were placed in the InAlyzer scanning area, whole-body scanning was performed, and scan images and body fat mass were measured using InAlyzer software.

For tissue observation, liver tissues extracted from each experimental group were cut into an appropriate size so that the lesion tissue area could be observed for hematoxylin and eosin staining (H&E staining), placed in a cassette, and fixed in 4% formalin solution. The fixed tissues were embedded in paraffin, cut into 4 μm sections, and stained with H&E. For Oil red O staining, livers extracted from each experimental group were cut into an appropriate size so that the lesion tissue area could be observed, and embedded in Tissue-Tek OCT compound (4583, Sakura Finetek, CA, USA). The embedded tissues were cut into 10 μm section using a cryostat (Leica CM1850, Wetzlar, Germany) and stained using Oil Red O stain. Stained sections were scanned using a Pannoramic 250 Flash III slide scanner (3DHistech, ltd., Budapest, Hungary) and captured using CaseViewer software.

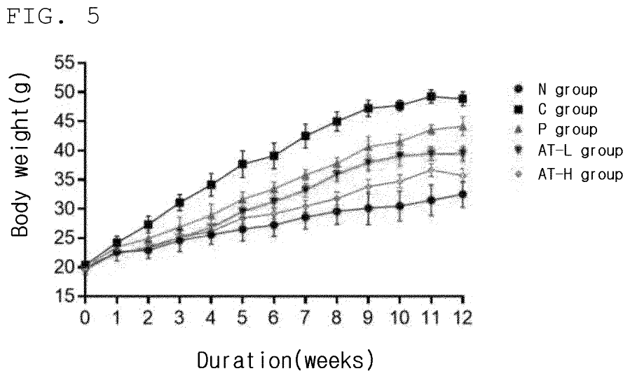

As a result, after 12 weeks, the body weight of mice in the high-fat diet group (C) (48.86±1.23 g) was significantly higher than that of mice in the normal group (N) (32.51±2.24 g), and the group that consumed Asimina triloba fruit extract (AT-L group and AT-H group) and the group that consumed the Garcinia cambogia extract (P) with known anti-obesity activity, showed a significant decrease in body weight compared to the C group (FIG. 5). In particular, the weight loss was greater in the AT-L group (39.45±3.30 g) and AT-H group (35.73±3.19 g) that consumed the Asimina triloba fruit extract than in the P group (44.17+1.59 g) that consumed the Garcinia cambogia extract, indicating a concentration-dependent weight loss effect of the Asimina triloba fruit extract.

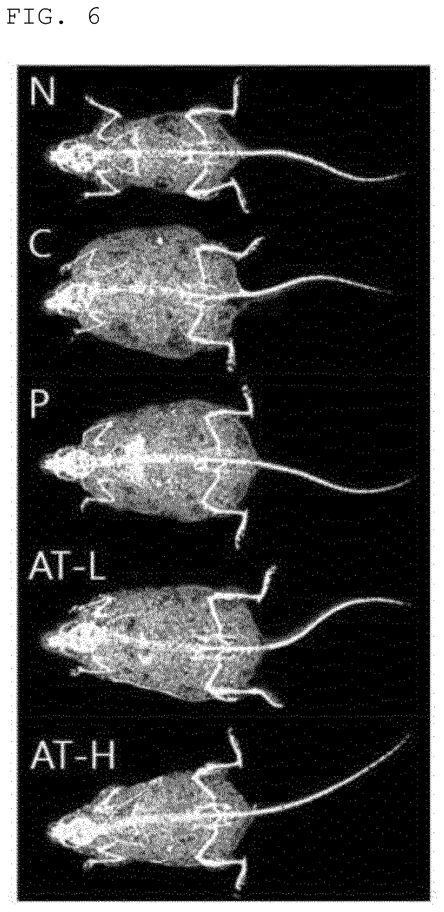

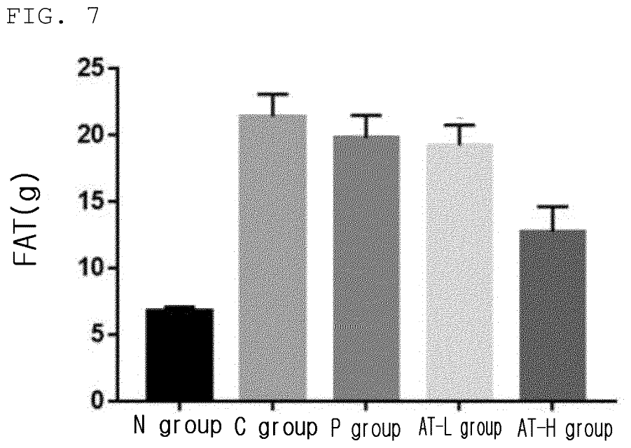

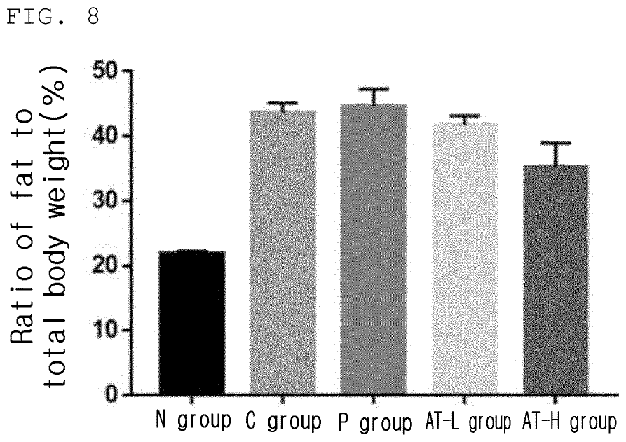

In the body composition analysis using InAlyzer (Medikors Inc., Seongnam, Korea), the body composition was measured by dual-energy X-ray absorptiometry and the image was analyzed. As a result, it was confirmed that the body fat was significantly reduced in the group that consumed the Asimina triloba extract (AT-L, AT-H group) compared to the high-fat diet group (C) (FIG. 6). Also, it was confirmed that in the AT-H group that consumed the Asimina triloba extract at a high concentration, the weight of adipose tissue (12.74±1.90 g) and a fat-to-weight ratio (35.24±3.71%) were significantly reduced compared to the high-fat diet group (C) and the group (P) that consumed Garcinia cambogia extract (FIGS. 7 and 8).

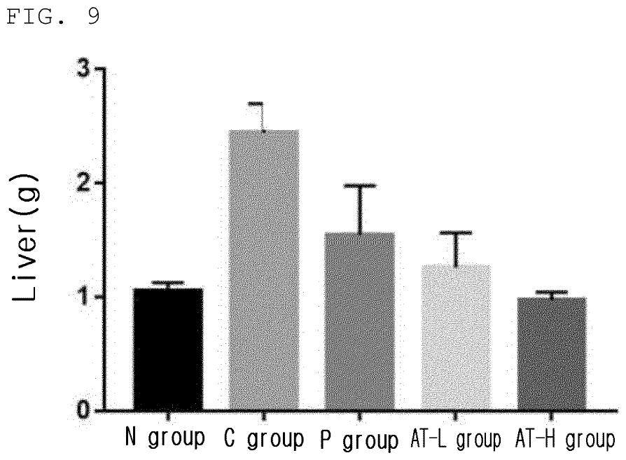

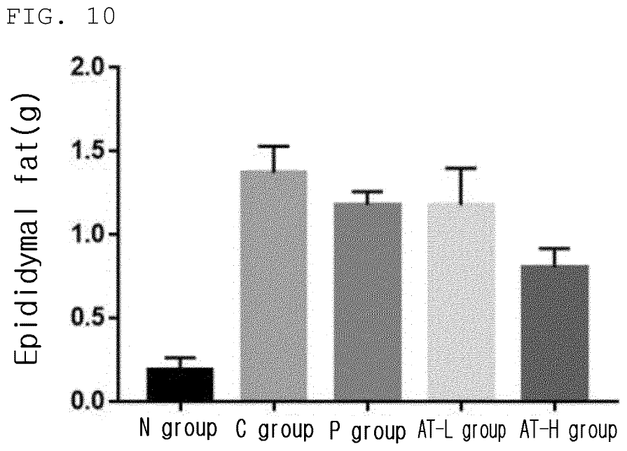

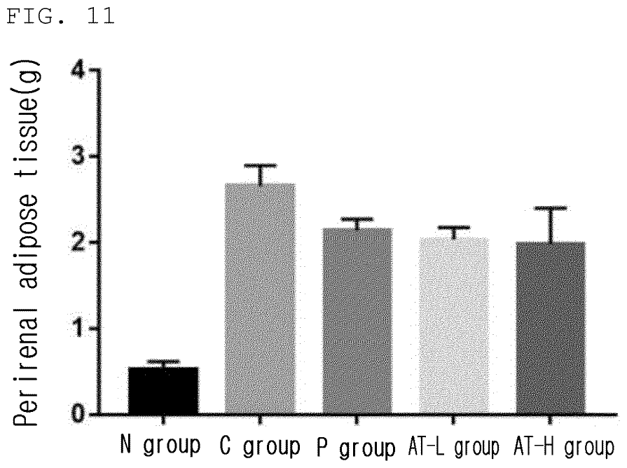

In addition, as a result of measuring the weight of liver, epididymal fat, and perirenal adipose tissue, it was confirmed that the weight of the C group (liver: 2.45±0.26 g, epididymal fat: 2.66±0.24 g, perirenal adipose: 1.37±0.16 g) was increased compared to the N group (liver: 1.06±0.07 g, epididymal fat: 0.53±0.09 g, perirenal adipose: 0.19±0.07 g). Also, it was confirmed that the weight of liver, epididymal fat, and perirenal adipose tissue was significantly reduced in the AT-L and AT-H groups that consumed the Asimina triloba fruit extract (FIGS. 9 to 11). Further, the AT-H group that consumed the Asimina triloba fruit extract at a high concentration showed a greater weight loss compared to the group that consumed the Asimina triloba fruit extract at a low concentration (AT-L).

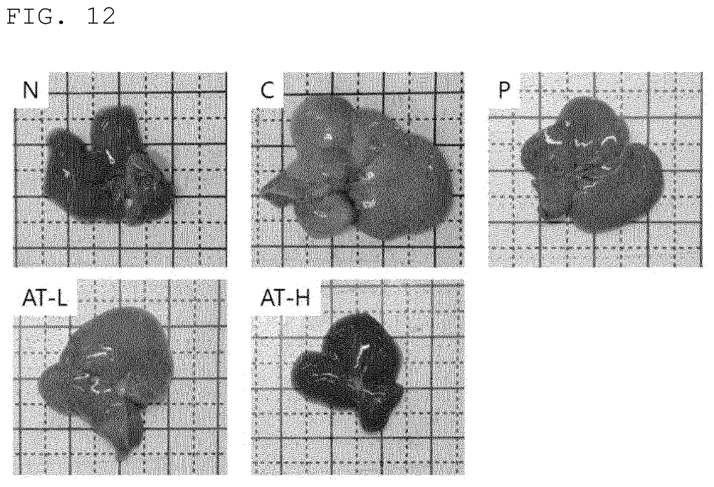

The livers of each experimental group were extracted and observed, and it was confirmed that the C group had an increase in liver size and fat deposition compared to the N group. Also, it was confirmed that the AT-H group that consumed that the Asimina triloba extract at a high concentration had a decrease in liver size and fat deposition, similar to the normal liver (N group) (FIG. 12).

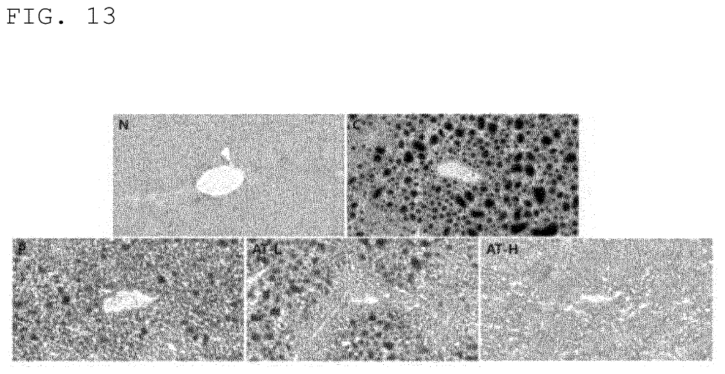

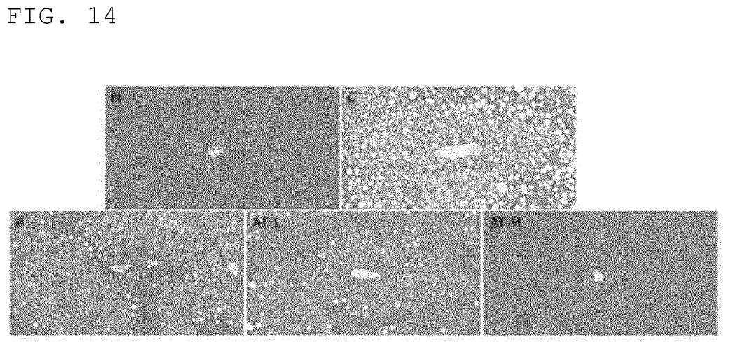

As a result of ORO staining of the liver tissue of each experimental group, ORO-stained lipid droplets were confirmed in the C group. Also, it was confirmed that the stained lipid droplets were significantly reduced in a concentration-dependent manner by consuming the Asimina triloba fruit extract (FIG. 13). Similar to the results of liver tissue ORO staining, liver tissue H&E experiment results also confirmed significantly increased fat deposition in the C group. Also, a significant decrease in fat deposition was confirmed in the groups that consumed the Asimina triloba fruit extract (AT-L and AT-H) (FIG. 14).

### 2-3. Blood Analysis

The blood of mice in each experimental group was analyzed to confirm the effect of the Asimina triloba extract on the inhibition of adipogenesis.

Specifically, after a 12-week animal experiment, blood was collected from mice anesthetized with isoflurane after a 12-hour fast. Blood was collected from the abdominal vein and centrifuged (2000× g, 15 minutes) to isolate the serum. The isolated serum was stored at −80° C. until it was used in the experiment, and total cholesterol, high-density lipoprotein (HDL), low-density lipoprotein (LDL), aspartate aminotransferase (AST), alanine aminotransferase (ALT), and lactate dehydrogenase (LDH) were analyzed.

Oral glucose tolerance tests for blood glucose measurement were performed at week 12 of the experiment after a 12-hour fast. Nine mice per group were orally administered 1 g/kg body weight of glucose, and blood glucose was measured using a glucometer (Accu-Chek; Roche Diagnostics, Indianapolis, IN, USA) at 0, 30, 60, 90, and 120 minutes.

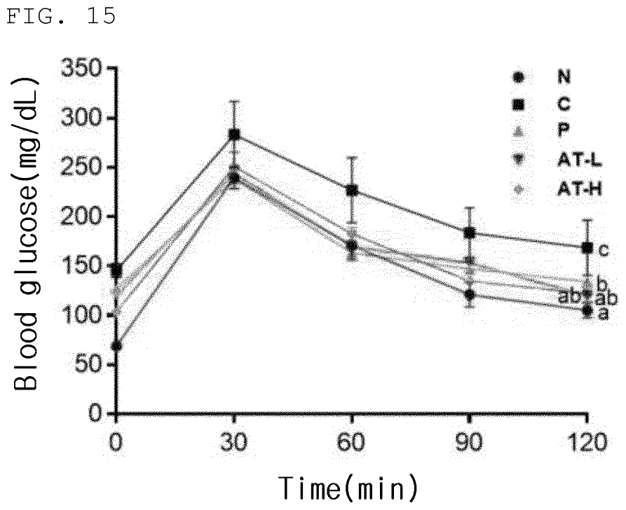

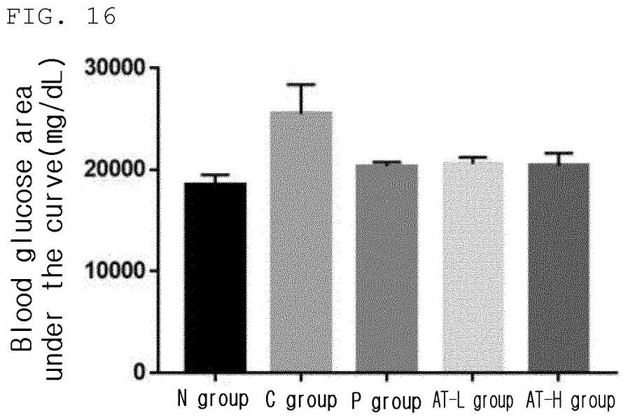

The results showed that blood glucose was the highest in the C group regardless of the time elapsed, and blood glucose was significantly lowered in the AT-L (119.83+19.42 mg/dL) and AT-H (121.67±21.79 mg/dL) groups that consumed the Asimina triloba fruit extract (FIG. 15). In addition, it was confirmed that the blood glucose area under the curve (AUC) was the highest in the C group as the control group, and was significantly lowered in the AT-L and AT-H groups that consumed the Asimina triloba fruit extract (FIG. 16).

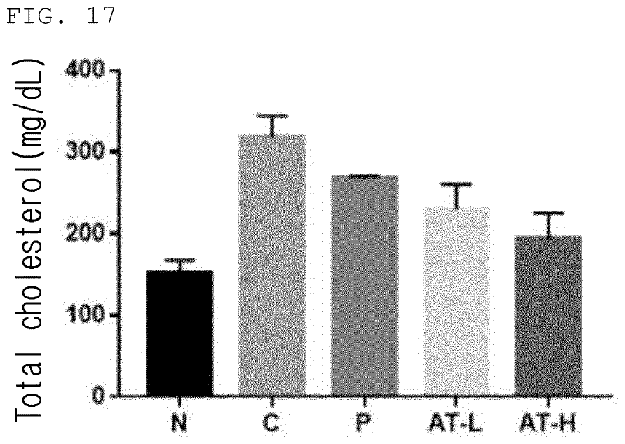

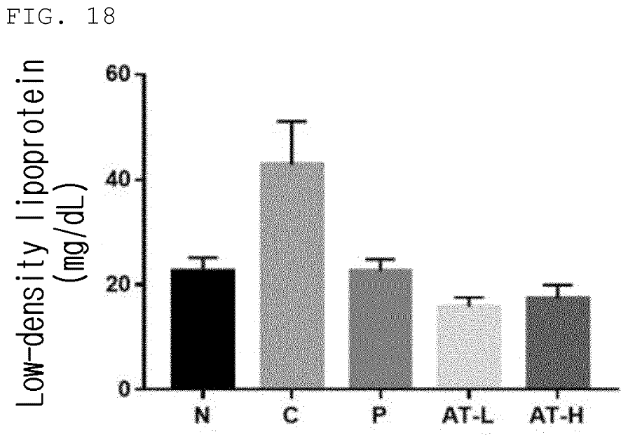

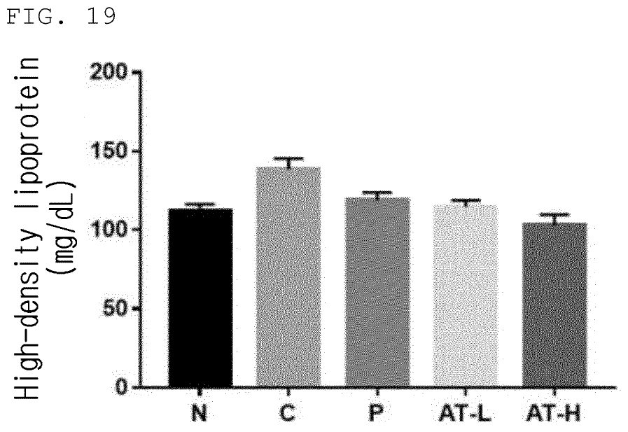

As a result of serum analysis, it was confirmed that total cholesterol (FIG. 17), low-density lipoprotein (FIG. 18), and high-density lipoprotein (FIG. 19), which are lipid-related markers, were the highest in the C group, and were decreased in the AT-L and AT-H groups that consumed the Asimina triloba fruit extract.

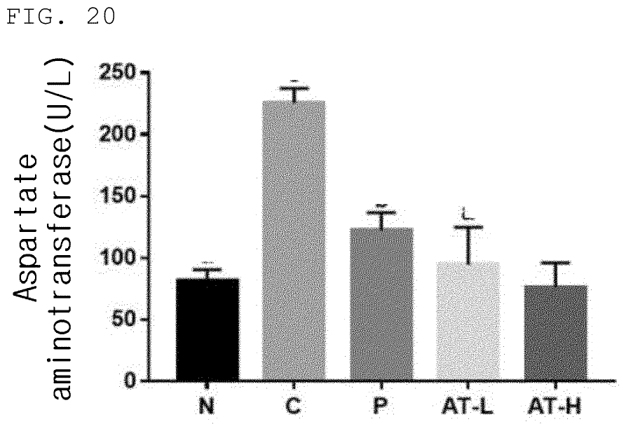

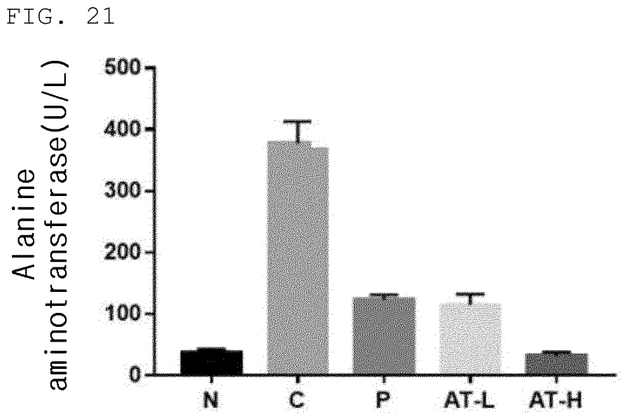

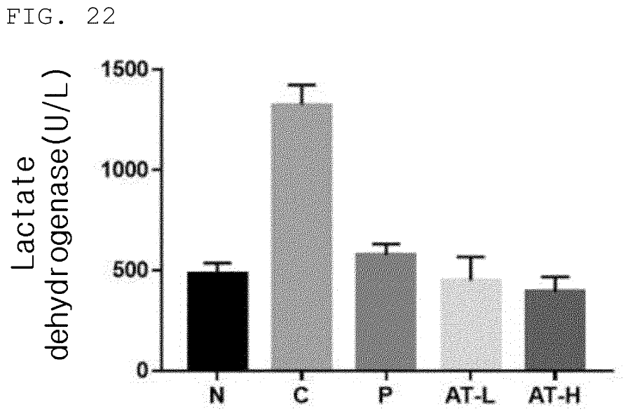

In addition, as a result of serum analysis of aspartate aminotransferase (AST) and alanine aminotransferase (ALT) that can check for liver damage, the increased AST and ALT levels in the C group were lowered by taking the Asimina triloba fruit extract, confirming that the Asimina triloba fruit extract was not hepatotoxic to the body (FIGS. 20 and 21). In addition, lactate dehydrogenase, which increases in blood concentration when cells are destroyed, was high in the C group, and the lactate dehydrogenase level was lowered by taking the Asimina triloba fruit extract, confirming that the Asimina triloba fruit extract has low toxicity (FIG. 22).

From the above description, those skilled in the art to which the present disclosure pertains will understand that the present disclosure can be implemented in other specific forms without changing its technical idea or essential features. In this regard, it should be understood that the embodiments described above are exemplary in all respects and are not intended to be limiting. The scope of the present disclosure should be interpreted that all changes or modifications derived from the meaning and scope of patent claims to be described below rather than the detailed description above and their equivalent concepts are included in the scope of the present disclosure.

1. A food composition for ameliorating or preventing obesity, lipid metabolism, or metabolic diseases, comprising an Asimina triloba extract as an active ingredient.

2. The food composition of claim 1, wherein the extract is one or more selected from the group consisting of seeds, fruits, roots, stems, and leaves of Asimina triloba.

3. The food composition of claim 1, wherein the extract is a crude extract obtained by extraction with a solvent selected from the group consisting of water, an alcohol having 1 to 4 carbon atoms, or a mixed solvent thereof.

4. The food composition of claim 1, wherein the extract is an extract obtained by extraction with a solvent selected from the group consisting of glycerin, butylene glycol, propylene glycol, methyl acetate, ethyl acetate, acetone, benzene, hexane, diethyl ether, dichloromethane, or a mixed solvent thereof.

5. The food composition of claim 1, wherein the metabolic disease is one or more selected from the group consisting of diabetes, diabetic complications, stroke, hyperlipidemia, fatty liver, arteriosclerosis, hypertension, cardiovascular diseases, or metabolic syndrome in which the above diseases occur simultaneously.

6. The food composition of claim 1, wherein the extract inhibits differentiation of preadipocyte cells and inhibits expression of transcription factors involved in adipogenesis and preadipocyte cell differentiation.

7. A pharmaceutical composition for preventing or treating obesity or metabolic diseases, comprising an Asimina triloba extract as an active ingredient.

8. The pharmaceutical composition of claim 7, wherein the extract is one or more selected from the group consisting of seeds, fruits, roots, stems, and leaves of Asimina triloba.

9. The pharmaceutical composition of claim 7, wherein the extract is a crude extract obtained by extraction with a solvent selected from the group consisting of water, an alcohol having 1 to 4 carbon atoms, or a mixed solvent thereof.

10. The pharmaceutical composition of claim 7, wherein the extract is an extract obtained by extraction with a solvent selected from the group consisting of glycerin, butylene glycol, propylene glycol, methyl acetate, ethyl acetate, acetone, benzene, hexane, diethyl ether, dichloromethane, or a mixed solvent thereof.

11. The pharmaceutical composition of claim 7, wherein the metabolic disease is one or more selected from the group consisting of diabetes, diabetic complications, stroke, hyperlipidemia, fatty liver, arteriosclerosis, hypertension, cardiovascular diseases, or metabolic syndrome in which the above diseases occur simultaneously.

12. The pharmaceutical composition of claim 7, wherein the extract inhibits differentiation of preadipocyte cells and inhibits expression of transcription factors involved in adipogenesis and preadipocyte cell differentiation.

13-22. (canceled)

23. A method for preventing or treating obesity or lipid metabolism, or metabolic disease, comprising administering to a subject a composition comprising an Asimina triloba extract as an active ingredient.

24-25. (canceled)
